(19) United States
(12) Patent Application Publication
(10) Pub. No.: US 2005/0008686 A1
(43) Pub. Date: Jan. 13, 2005

(54) Cochleate preparations of fragile nutrients

(76) Inventors: Raphael J. Mannino, Annandale, NJ (US); Sara L. Krause-Elsmore, Kearny, NJ (US); Susan Gould-Fogerite, Annandale, NJ (US); David Delmarre, Jersey City, NJ (US); Feng Tan, Newark, NJ (US)

Correspondence Address: LAHIVE & COCKFIELD, LLP., 28 STATE STREET, BOSTON, MA, 02109, US

(21) Appl. No.: 10/759,381

(22) Filed: Jan. 15, 2004

Related U.S. Application Data
(60) Provisional application No. 60/440,120, filed on Jan. 15, 2003.
(60) Provisional application No. 60/465,754, filed on Apr. 25, 2003.

(51) Int. Cl. A61K 9/127

(52) U.S. Cl. 424/450

(57) ABSTRACT

The present invention provides novel fragile nutrient cochleates that generally include one or more encochleated fragile nutrients, e.g., beta carotene, and methods for making the same. Also provided are methods for the use and administration of the fragile nutrient cochleates of the invention.

### RELATED APPLICATIONS

This application claims the benefit of U.S. Provisional Application Nos. 60/440,120, filed Jan. 15, 2003, and 60/465,754, filed Apr. 25, 2003, which applications are incorporated herein by this reference.

### FIELD OF THE INVENTION

The present invention relates generally to cochleate preparations of fragile nutrients, and methods of forming and administering the same.

### BACKGROUND OF THE INVENTION

Cochleate structures, as an intermediate in the preparation of large unilamellar vesicles, have been described in U.S. Pat. No. 4,078,052. Liposomal and cochleate oral delivery vehicles are known and have been disclosed, e.g., in U.S. Pat. No. 5,994,318, entitled “Cochleate Delivery Vehicles,” the entire disclosure of which is incorporated in its entirety by reference herein. U.S. Pat. No. 5,994,318 discloses, e.g., nutrient cochleates including vitamin A cochleates, lipophilic drug cochleates, pigment cochleates, saccharide cochleates, enzyme cochleates, adjuvant cochleates, and methods of cochleate manufacture. Alternative methods of forming cochleates using aqueous two-phase systems also have been described, e.g., in U.S. Pat. No. 6,153,217.

### SUMMARY OF THE INVENTION

The present invention provides novel fragile nutrient cochleates and methods of manufacture. The fragile nutrient cochleates of the present invention are stable and capable of delivering desired amounts of fragile nutrients in an active state. The present invention is based, at least in part, on the discovery that fragile nutrients can be efficiently and stably incorporated into cochleates at larger concentrations achievable with conventional methods of cochleate formation by dissolution of both the fragile nutrient and the lipid in a solvent prior to formation of cochleates. Moreover, the fragile nutrient cochleates can be processed and stored, over a large range of temperatures, pressures and shear without degradation of the fragile nutrient.

In one aspect, the present invention provides a fragile nutrient cochleate formulation including a fragile nutrient component, and a cochleate including a negatively charged lipid component and a multivalent cation component. In some embodiments, the fragile nutrient cochleate formulation contains at least about 1% fragile nutrient. Preferably, the fragile nutrient cochleate formulation contains at least about 3% fragile nutrient, and more preferably at least about 5% fragile nutrient. The negatively charged lipid component can include phosphatidylserine, e.g., soy phosphatidylserine. In a preferred embodiment, the fragile nutrient is beta-carotene and the formulation further comprises a vitamin E component.

The fragile nutrient can be a phytochemical, e.g., an antioxidant phytochemical or a zoochemical. The phytochemical can be beta-carotene, lutein, zeaxanthine, quercetin, silibin, perillyl alcohol, genistein, sulfurophane and lycopene. The zoochemical can be an omega-3 or omega-6 fatty acid. The cochleate can optionally contain one or more additional cargo moieties, e.g., a vitamin, a mineral, a nutrient, a micronutrient, an arnino acid, a toxin, a microbicide, a microbistat, a co-factor, an enzyme, a polypeptide, a polypeptide aggregate, a polynucleotide, a lipid, a carbohydrate, a nucleotide, a starch, a pigment, a fatty acid, a monounsaturated fatty acid, a polyunsaturated fatty acid, a flavor substance, a flavored essential oil or extract, a hormone, a cytokine, a virus, an organelle, a steroid or other multi-ring structure, a saccharide, a metal, a metabolic poison, an antigen, an imaging agent, a porphyrin, a tetrapyrrolic pigment, and a drug.

In another aspect, the present invention provides a food item or a personal care product including a fragile nutrient cochleate. The food item can be a health bar, snack food, domesticated animal food, fish food, poultry feed, dog food, cat food, animal food, or a health drink. The personal care item can be a hair care product or a skin care product.

In another aspect, the present invention provides a pharmaceutical composition which includes a fragile nutrient cochleate formulation.

In another aspect, the invention provides a method of making a fragile nutrient cochleate formulation. The method includes the steps of: dissolving a lipid component and a fragile nutrient in an organic solvent to form a solution; forming fragile nutrient liposomes from the solution; and exposing the fragile nutrient-liposomes to cation to form fragile nutrient cochleates. The step of forming the fragile nutrient liposomes can include the addition of salt water to the solution. The step of forming the fragile nutrient liposomes can further include removal of the solvent from the solution to form a film. In one embodiment, the solvent includes tetrahydrofuran, chloroform, dichloromethane, carbon tetrachloride, butanol, hexane, ethanol, toluene, benzene, ether, petrol ether, oil or combinations thereof.

The method can include the step of adding fragile nutrient cochleates to a food item or a personal care product. The food item can be a health bar, snack food, domesticated animal food, fish food, poultry feed, dog food, cat food, animal food, or health drink. The personal care item can be a hair care product or a skin care product.

In yet another aspect, the invention provides a method of delivering a fragile nutrient to a subject comprising administering to a subject a biologically effective amount of fragile nutrient cochleate. The fragile nutrient cochleate can be delivered in the form of a food item. The food item can be a health bar, snack food, dog food, cat food, animal food, or health drink.

The fragile nutrient cochleate can also be administered mucosally, systemically, orally, intranasally, intraocularly, intrarectally, intravaginally, intrapulmonarily, intravenously, intramuscularly, subcutaneously, transdermally or intradermally in order to treat inflammation, pain, infection, fungal infection, bacterial infection, viral infection, parasitic disorders, an immune disorder, genetic disorders, degenerative disorders, cancer, diabetes, insomnia, proliferative disorders, obesity, depression, hair loss, impotence, hypertension, hypotension, dementia, senile dementia, or malnutrition.

These and other objects, along with advantages and features of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings, in which:

FIG. 1 illustrates an exemplary method for making the fragile nutrient cochleates of the present invention.

FIG. 2 and FIG. 3 are images of beta-carotene cochleates in various stages of manufacture, including beta-carotene in solution, incorporated into liposomes, incorporated into cochleates, and upon release from the cochleate.

FIG. 4 illustrates yet another method for making fragile nutrient cochleates of the present invention that eliminates the solvent film step of the method described in FIG. 2.

FIG. 5 illustrates a method of making beta-carotene and vitamin E cochleates in accordance with the invention.

FIG. 6 illustrates another method of making beta-carotene and vitamin E cochleates in accordance with the invention.

FIG. 7 is two images of fish oil cochleates prepared in accordance with the present invention before (left image) and after (right image) addition of chelators.

### DETAILED DESCRIPTION OF THE INVENTION

The novel cochleate preparations of the present invention provide an efficient, stable, safe, and healthy delivery means for fragile nutrients such as phytochemicals, zoochemicals, and antioxidants. The invention also provides the ability to deliver improved potency and therefore enhanced health benefits whether used in processed food, beverages personal care products, or in conventional treatment methods, e.g., tablets or pills, and whether intended for humans or other animals, such as livestock (e.g., cattle, horses, farm-raised fish, and chickens) and/or companion animals (e.g., dogs, cats, birds, rodents, and fish). The fragile nutrient cochleates of the invention can be added directly to food as a dietary supplement, incorporated into food for consumption (e.g., health bars and drinks), taken individually (e.g., in a capsule or beverage), taken in conjunction with prescription or over the counter medicaments, incorporated into a personal care product for topical application, and/or delivered non-orally (e.g., by injection, patch and suppository).

In order to more clearly and concisely describe the subject matter of the claims, the following definitions are intended to provide guidance as to the meaning of specific terms used in the following written description, examples and appended claims.

As used herein, the term “fragile nutrients” refers to fragile compounds (e.g., susceptible to degradation by oxygen, water and the like) derived from plant sources (phytochemicals), animal sources (zoochemicals), or synthetic sources that are either known or are suspected of contributing to the health of an animal. Examples of fragile nutrients include, but are not limited to, beta-carotene, lutein, zeaxanthine, quercetin, silibin, perillyl alcohol, genistein, sulfurophane, lycopenes, and essential fatty acids, including eicosapentanoic acid (EPA), gamma-3, omega-3, gamma-6, and omega-6 fatty acids.

As used herein, the term “anti-oxidants” refers to compounds that are able to cancel out or react with oxidative species such as singlet oxygen or oxidative free radicals so as to protect vital subcellular components (e.g., DNA) from damage.

As used herein, the term “essential nutrients” refers to nutrients such as fatty acids that must be derived from the diet because of the inability to synthesize these nutrients in vivo for lack of the required enzymes.

As used herein, “micronutrient” is a nutrient that the body must obtain from outside sources. Generally micronutrients are essential to the body in small amounts.

As used herein, the terms “cochleate” and “precipitate” are used interchangeably to refer to lipid precipitates that include alternating cationic and lipid bilayer sheets stacked and/or rolled up with little or no internal aqueous space, wherein the cationic sheet is comprised of one or more multivalent cations. Additionally, the term “encochleated” means associated with the cochleate structure, e.g. by incorporation into the cationic sheet, and/or inclusion in the lipid bilayer. As used herein, the term “food” refers to any object or objects suitable for consumption by a human or non-human animal.

The term “delivery,” as used herein, refers to any means of bringing or transporting a cargo moiety and/or fragile nutrient to a host, a food item, a formulation, a pharmaceutical composition, or any other system, wherein the cargo moiety and/or fragile nutrient maintains at least a portion of its activity

The term “cargo moiety” refers to any compound having a property of biological interest, e.g., one that has a role in the life processes of a living organism, and generally does not refer to the lipid and ion employed to form the cochleate. A cargo moiety may be organic or inorganic, a monomer or a polymer, endogenous to a host organism or not, naturally occurring or synthesized in vitro and the like. Thus, examples include, vitamins, minerals, nutrients, micronutrients, amino acids, toxins, microbicides, microbistats, co-factors, enzymes, polypeptides, polypeptide aggregates, polynucleotides, lipids, carbohydrates, nucleotides, starches, pigments, fatty acids, monounsaturated fatty acids, polyunsaturated fatty acids, flavorings, essential oils or extracts, hormones, cytokines, viruses, organelles, steroids and other multi-ring structures, saccharides, metals, metabolic poisons, antigens, imaging agents, porphyrins, tetrapyrrolic pigments, drugs and the like.

The lipid employed in the present invention preferably includes one or more negatively charged lipids. As used herein, the term “negatively charged lipid” includes lipids having a head group bearing a formal negative charge in aqueous solution at an acidic, basic or physiological pH, and also includes lipids having a zwitterionic head group.

The term “multivalent cation,” refers to a divalent cation or higher valency cation. Examples of multivalent cations include, but are not limited to, calcium, magnesium, barium, zinc, iron and other elements capable of forming ions or other structures having multiple positive charges capable of chelating and bridging negatively charged lipids. Additionally or alternatively, the multivalent cation can include other multivalent cationic compounds, e.g., cationic cargo moieties.

In its broadest aspects, the present invention provides the skilled artisan with the methods and information sufficient to make a stable preparation of fragile micronutrients. Guidance provided herein will facilitate the inclusion of such fragile nutrients in various preparations (e.g., in pharmaceutical preparations and over the counter preparations) for the delivery of the fragile nutrients, in an active state, to living systems.

One advantage of the present invention is that employing the methods of the invention, fragile nutrients can be incorporated into a cochleate structure more efficiently than cochleates manufactured employing conventional methods. Cochleate formulations of the invention preferably contain at least about 1% fragile nutrient by weight. More preferably, cochleate formulations of the invention contain at least about 2%, 3%, 4%, 5%, 8%, 10%, 20%, 30%, 50% . . . 90% fragile nutrient by weight. Cochleates including one more fragile nutrients in amounts in all ranges and values between 0.5% and 99% are within the scope of the present invention.

Another advantage of the present invention is that the fragile nutrients are substantially incorporated into the cochleate structure. As used herein, the term “substantially incorporated” means that a substantial amount of the fragile nutrient component is within the cochleate, i.e., not projecting from the cochleate surface. Preferably at least 20%, 30%, 50%, 70%, 90%, 95%, or even 99% of the fragile nutrient is incorporated into the cochleate structure.

Yet another advantage of the present invention is that the cochleates of the invention offer higher incorporation of fragile nutrients in comparison to conventionally prepared cochleates. In some embodiments, the cochleates of the invention incorporate 10%, 20%, 30%, 50%, 75%, 100%, 500%, 1000% and even 10000% more fragile nutrient component than conventional cochleates.

Yet another advantage of the present invention is the ability of the cochleates to mask tastes and/or odors. The present invention provides a means for masking flavors and odors, such as those associated with omega-3fatty acids, by substantial incorporation within a cochleate structure.

Omega-3fatty acid cochleates can be used in goods that are consumed without noticable taste or odor. Omega-3fatty acids are found mainly in fish oils and other fish products, and typically exhibit a fish-like odor and a greenish tint. Omega-3 fatty acids have been implicated in increased disease resistance and fertility in animals, and they are shown to have a significantly positive effect on cholesterol and overall cardiovascular health in human beings. See, for example, Daviglus et al. N Engl J Med. 336: 1046-1053 (1997). One of the complications of incorporating them directly into food, however, is their noticeable odor and taste. The present invention addresses this disadvantage by masking the odor and taste associated with fish oil.

The present invention is also particularly advantageous for the delivery of antioxidant fragile nutrients such as beta-carotene. Beta-carotene acts as an antioxidant by quenching singlet oxygen and other free radicals. Unfortunately beta-carotene and other carotenoids are highly susceptible to oxidation prior to incorporation into the body. This phenomenon is observed as a bleaching of the deep orange color. Britton, FASEB J. 9: 1551-1558 (1995).

Incorporation of the fragile nutrient into the cochleates of the invention also is advantageous because it provides the fragile nutrient with protection from both the environment, e.g., water and oxygen, and also the stomach. For example, the present invention provides beta-carotene with an oxygen-free environment for storage before use. The activity is indicated by the intensity of the red-orange color of the beta-carotene. Beta-carotene cochleates of the present invention have been observed to retain their intense color despite exposure to extreme environmental stresses.

The present invention is also advantageous because the resultant fragile nutrient cochleate formulations are highly stable, e.g., they can withstand extreme temperature, high relative humidity and pressure. Fragile nutrient cochleate formulations of the present invention can preferably retain substantially the same fragile nutrient activity 20%, 30%, 50%, 100% and even 1000% longer than unprotected fragile nutrient, at the same temperature and relative humidity. Fragile nutrient cochleate formulations of the present invention also can preferably retain substantially the same fragile nutrient activity 20%, 30%, 50%, 100% and even 1000% longer than fragile nutrients encochleated using standard methods, under equivalent environmental conditions such as the same temperature and relative humidity. Preferably, the fragile nutrient cochleates of the present invention retain substantially the same fragile nutrient activity as a fresh composition for at least 3 months, 6 months, 1 year, 2 years, 3 years, 5 years or even 10 years from formulation.

In one aspect, the invention features fragile nutrient cochleate formulations that include one or more fragile nutrient components (e.g., beta-carotene), and a cochleate that includes a negatively charged lipid component and a multivalent cation component.

Suitable fragile nutrients include phytochemicals, zoochemicals, and antioxidants. Examples of such fragile nutrients include, but are not limited to, beta-carotene, lutein, zeaxanthine, quercetin, silibin, perillyl alcohol, genistein, sulfurophane, lycopenes, and essential fatty acids, including eicosapentanoic acid (EPA), gamma-3, omega-3, gamma-6, and omega-6 fatty acids.

Of particular interest are fragile nutrients such as beta-carotene, also known a pro-vitamin A. Cartenoids, including beta-carotene, are the precursors of vitamin A (a member of the retinoid family). Sources rich in beta-carotene are green plants (e.g., grasses, clover and carrots). Most other cereals and vegetables, however, contain little or no beta-carotene. Moreover, even in plants originally rich in beta-carotene, over the growing season and upon conventional processing the beta-carotene levels decrease, so that animals must be fed vitamin supplements or fresh greens to obtain beta-carotene. Beta-carotene is unstable when exposed to light and air, and typically is stored under nitrogen and under nitrogen at 0° C. to maintain its stability.

The present invention is based, at least in part, on the discovery that one or more solvents such as tetrahydrofuran (THF), can be employed to form an improved cochleate with fragile nutrients such as beta-carotene at desirable concentrations. Suitable solvents and solvent mixtures that can be employed in connection with the present invention and can readily be identified by a person of skill in the art employing the teachings provided herein and knowledge in the art. For example, beta-carotene was added to a variety of potential solvents, and was found not soluble in either water or DMSO, and slightly soluble in methyl pyrrolidone (less than 1 mg/ml). Heptane also was considered, but heptane is not miscible with water and thus cannot readily be employed in the formation of cochleates. Surprisingly however, THF was found to solubilize 10 mg/ml of beta-carotene. Similarly, solvents can be identified for other fragile nutrients and lipids in accordance with the present invention.

The solvent or solvents selected preferably are organic solvents. Preferably, the solvent is an FDA acceptable solvent. Examples of suitable solvents include, but are not limited to tetrahydrofuran, chloroform, dichloromethane, carbon tetrachloride, butanol, hexane, ethanol, toluene, benzene, ether, petrol ether, oil or combinations thereof. THF is particularly advantageous because it is safer than conventional solvents used to form cochleates and liposomes, such as chloroform. In addition, mixtures of solvents can be employed in accordance with the present invention. Solvent mixtures can be useful, for example, for when the lipid is more readily soluble in one solvent and the fragile nutrient is more readily soluble in another; the solvents can be mixed before or after solubilizing the lipid and fragile nutrient in each.

The lipid component can include one or more negatively charged phospholipids, e.g., and phosphatidylserine, phosphatidylinositol, phosphatidic acid and/or phosphatidylglycerol and/or a mixture of one or more of these lipids with other lipids. Additionally or alternatively, the lipid can include phosphatidylcholine (PC), phosphatidylethanolamine (PE), diphosphotidylglycerol (DPG), and the like.

In one embodiment, the lipid is a mixture of lipids, comprising at least 75% negatively charged lipid. In another embodiment, the lipid includes at least 85% negatively charged lipid. In other embodiments, the lipid includes at least 90%, 95% or even 99% negatively charged lipid. All ranges and values between 60% and 100% negatively charged lipid are meant to be encompassed herein.

The lipids can be natural or synthetic. For example, the lipid can include esterified fatty acid acyl chains, or organic chains attached by non-ester linkages such as ether linkages (as described in U.S. Pat. No. 5,956,159), disulfide linkages, and their analogs.

In one embodiment the lipid chains are from about 6 to about 26 carbon atoms, and the lipid chains can be saturated or unsaturated. Fatty acyl lipid chains useful in the present invention include, but are not limited to, n-tetradecanoic, n-hexadecanoic acid, n-octadecanoic acid, n-eicosanoic acid, n-docosanoic acid, n-tetracosanoic acid, n-hexacosanoic acid, cis-9-hexadecenoic acid, cis-9-octadecenoic acid, cis,cis-9,12- octadecedienoic acid, all-cis-9,12,15-octadecetrienoic acid, all-cis-5,8,11,14-eicosatetraenoic acid, all-cis-4,7,10,13,16,19-docosahexaenoic acid, 2,4,6,8-tetramethyl decanoic acid, and lactobacillic acid, and the like.

In some embodiments, pegylated lipid also is included. Pegylated lipid includes lipids covalently linked to polymers of polyethylene glycol (PEG). PEG's are conventionally classified by their molecular weight, thus PEG 6,000 MW, e.g., has a molecular weight of about 6000. Adding pegylated lipid generally will result in an increase of the amount of compound (e.g., peptide, nucleotide, and nutrient) that can be incorporated into the precipitate. An exemplary pegylated lipid is dipalmitoylphosphatidylehtanolamine (DPPE) bearing PEG 5,000 MW.

The negatively charged lipid can include soy-based lipids. In a preferred embodiment, the negatively charged lipid component is soy phosphatidylserine. One skilled in the art can determine readily how much lipid must be negatively charged by preparing a mixture with known concentrations of negative and non-negative lipids and by any of the procedures described herein, determining whether precipitates form.

The multivalent cation can be any multivalent cation that can induce the formation of a cochleate or other lipid precipitate. Examples of suitable cations include calcium, magnesium, barium, zinc, iron and/or a cationic cargo moiety. Cochleates made with different cations have different structures and convert to liposomes at different rates. Because of these structural differences, the rate of release of the fragile nutrient contained therein varies. Accordingly, by combining cochleates made with different cations, formulations that will release the fragile nutrient over a protracted period of time are obtainable.

The cochleates of the instant invention also serve as excellent means for delivering additional cargo moieties to a host. Because the cargo moiety is substantially incorporated into the cochleate, in a non-aqueous environment, the cargo moiety also is stabilized and preserved. This also can be advantageous when it is desired to deliver both vitamins and minerals, as conventional preparations of vitamins and minerals often produced discoloration and/or metallic tastes to the preparations they are added to.

Accordingly, the formulations of the present invention can optionally include additional cargo moieties (i.e., cargo moieties in addition to the fragile nutrient). In one embodiment, the additional cargo moiety is another fragile nutrient. In a preferred embodiment, the cargo moiety is a vitamin (e.g., vitamin E), or a mineral (e.g., zinc).

Suitable additional cargo moieties also include vitamins, minerals, nutrients, micronutrients, amino acids, toxins, microbicides, microbistats, co-factors, enzymes, polypeptides, polypeptide aggregates, polynucleotides, lipids, carbohydrates, nucleotides, starches, pigments, fatty acids, monounsaturated fatty acids, polyunsaturated fatty acids, flavorings, essential oils or extracts, hormones, cytokines, viruses, organelles, steroids and other multi-ring structures, saccharides, metals, metabolic poisons, antigens, imaging agents, porphyrins, tetrapyrrolic pigments, drugs and the like.

The cargo moiety can be a diagnostic agent, such as an imaging agent. Imaging agents include nuclear agents and porphyrins (e.g., tetrapyrrolic agents or pigments such as Zinc Tetra-Phenyl Porphyrin).

The polynucleotide can be one that is expressed to yield a biologically active polypeptide or polynucleotide. Thus, the polypeptide may serve as an immunogen or, for example, have enzymatic activity. The polynucleotide may have catalytic activity, for example, be a ribosome, or may serve as an inhibitor of transcription or translation, e.g., a an antisense molecule. If expressed, the polynucleotide preferably includes the necessary regulatory elements, such as a promoter, as known in the art. A specific example of a polypeptide is insulin.

The cargo moiety can be an organic molecule that is hydrophobic in aqueous media. The cargo moiety can also be a water-soluble polyvalent cationic molecule.

The drug can be, but is not limited to, a protein, a small peptide, a bioactive polynucleotide, an antibiotic, an antiviral, an anesthetic, an anti-infectious, an antifungal, an anticancer, an immunosuppressant, a steroidal anti-inflammatory, a non-steroidal anti-inflammatory, an antioxidant, an antidepressant which can be synthetically or naturally derived, a substance which supports or enhances mental function or inhibits mental deterioration, an anticonvulsant, an HIV protease inhibitor, a non-nucleophilic reverse transcriptase inhibitor, a cytokine, a tranquilizer or a vasodilatory agent. The drug can also be any over the counter (non-prescription) medication.

Examples of suitable drugs include Amphotericin B, acyclovir, adriamycin, carbamazepine, ivermectin, melphalen, nifedipine, indomethacin, curcumin, aspirin, ibuprofen, naproxen, acetaminophen, rofecoxib, diclofenac, ketoprofin, meloxicam, nabumetone, estrogens, testosterones, steroids, phenytoin, ergotamines, cannabinoids, rapamycin, propanadid, propofol, alphadione, echinomycin, miconazole nitrate, teniposide, hexamethylmelamine, taxol, taxotere, 18-hydorxydeoxycorticosterone, prednisolone, dexamethazone, cortisone, hydrocortisone, piroxicam, diazepam, verapamil, tobramycin, caspofungin, geldanamycin, nystatin, rifampin, tyrphostin, a glucan synthesis inhibitor, vitamin A acid, mesalamine, risedronate, nitrofurantoin, dantrolene, etidronate, nicotine, amitriptyline, clomipramine, citalopram, dothepin, doxepin, fluoxetine, imipramine, lofepramine, mirtazapine, nortriptyline, paroxetine, reboxitine, sertraline, trazodone, venlafaxine, dopamine, St. John's wort, phosphatidylserine, phosphatidic acid, amastatin, antipain, bestatin, benzamidine, chymostatin, 3,4-dichloroisocoumarin, elastatinal, leupeptin, pepstatin, 1,10-phenanthroline, phosphoramidon, ethosuximide, ethotoin, felbamate, fosphenytoin, lamotrigine, levitiracetam, mephenytoin, methsuximide, oxcarbazepine, phenobarbital, phensuximide, primidone, topirimate, trimethadione, zonisamide, saquinavir, ritonavir, indinavir, nelfinavir, and amprenavir.

The drug can be a polypeptide such as cyclosporin, angiotensin 1, II and III, enkephalins and their analogs, ACTH, anti-inflammatory peptides I, II, III, bradykinin, calcitonin, b-endorphin, dinorphin, leucokinin, leutinizing hormone releasing hormone (LHRH), insulin, neurokinins, somatostatin, substance P, thyroid releasing hormone (TRH) and vasopressin.

The drug can be an antigen, but is not limited to a protein antigen. The antigen can also be a carbohydrate or DNA. Examples of antigenic proteins include membrane proteins, carbohydrates, envelope glycoproteins from viruses, animal cell proteins, plant cell proteins, bacterial proteins, and parasitic proteins.

The antigen can be extracted from the source particle, cell, tissue, or organism by known methods. Biological activity of the antigen need not be maintained. However, in some instances (e.g., where a protein has membrane fusion or ligand binding activity or a complex conformation which is recognized by the immune system), it is desirable to maintain the biological activity. In these instances, an extraction buffer containing a detergent which does not destroy the biological activity of the membrane protein is employed. Suitable detergents include ionic detergents such as cholate salts, deoxycholate salts and the like or heterogeneous polyoxyethylene detergents such as Tween, BRIG or Triton.

Suitable nutrients include, but are not limited to vitamins, minerals, fatty acids, amino acids, fish oils, fish oil extracts, and saccharides, vitamins, herbal products, essential oils or minerals. Specific examples include Vitamins A, B, B1, B2, B3, B12, B6, B-complex, C, D, E, and K, herbs, spices, and iron. Minerals include, but are not limited to boron, chromium, colloidal minerals, colloidal silver, copper, manganese, potassium, selenium, vanadium, vanadyl sulfate, calcium, magnesium, barium, iron and zinc.

The cargo moiety can be a saccharide or sweetener, e.g., saccharine, isomalt, maltodextrine, aspartame, glucose, maltose, dextrose, fructose and sucrose. Flavor agents include oils, essential oils, or extracts, including but not limited to oils and extracts of cinnamon, vanilla, almond, peppermint, spearmint, chamomile, geranium, ginger, grapefruit, hyssop, jasmine, lavender, lemon, lemongrass, marnjoramn, lime, nutmeg, orange, rosemary, sage, rose, thyme, anise, basil, and black pepper tea or tea extracts, an herb, a citrus, a spice or a seed.

Another aspect of the present invention is a method of making a fragile nutrient cochleate formulation. The method includes the steps of: (i) dissolving a lipid component and a fragile nutrient component in an organic solvent (e.g., THF) to form a solution; (ii) forming fragile nutrient liposomes; and (iv) exposing the liposomes to cation to form fragile nutrient cochleates.

The solvent can optionally be removed prior to the formation of liposomes and/or after the liposomes are formed. Any known solvent removal method can be employed. For example, solvent may be removed from the liposomal suspension by tangential flow and/or filtration and/or dialysis, or from the lipid-fragile nutrient solution by drying under a stream of nitrogen to form a film. Removal of the solvent may be advantageous because the solvent creates a favorable environment in which the fragile nutrient resides. Removing the favorable environment would facilitate the incorporation of the fragile nutrient into the cochleate structure.

Fragile nutrient liposomes can be formed by adding the lipid-fragile nutrient solution to an aqueous solution. Additionally or alternatively, the lipid-fragile nutrient can be agitated with an aqueous solution in order to form liposomes. The aqueous solution is preferably salt water. Salt water is highly polar, and creates an unfriendly environment for the fragile nutrient. The fragile nutrient, therefore, would be forced into the cochleate structure.

Without wishing to be bound by any particular theory, it is believed that the ability to dissolve the fragile nutrient, i.e., the smaller size of the fragile nutrient to be encochleated, facilitates the incorporation of the fragile nutrient into the cochleate structure.

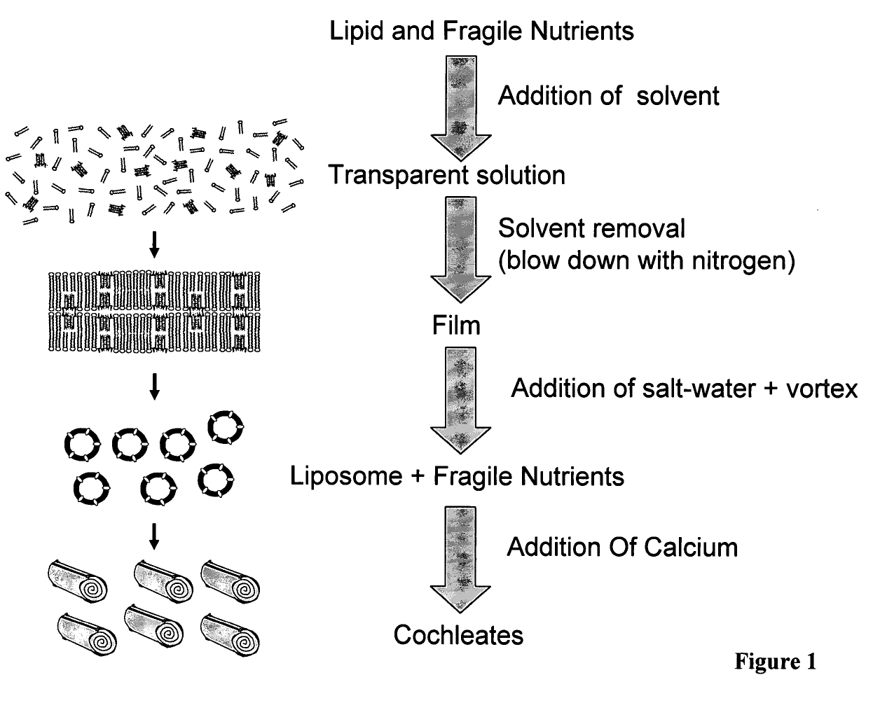

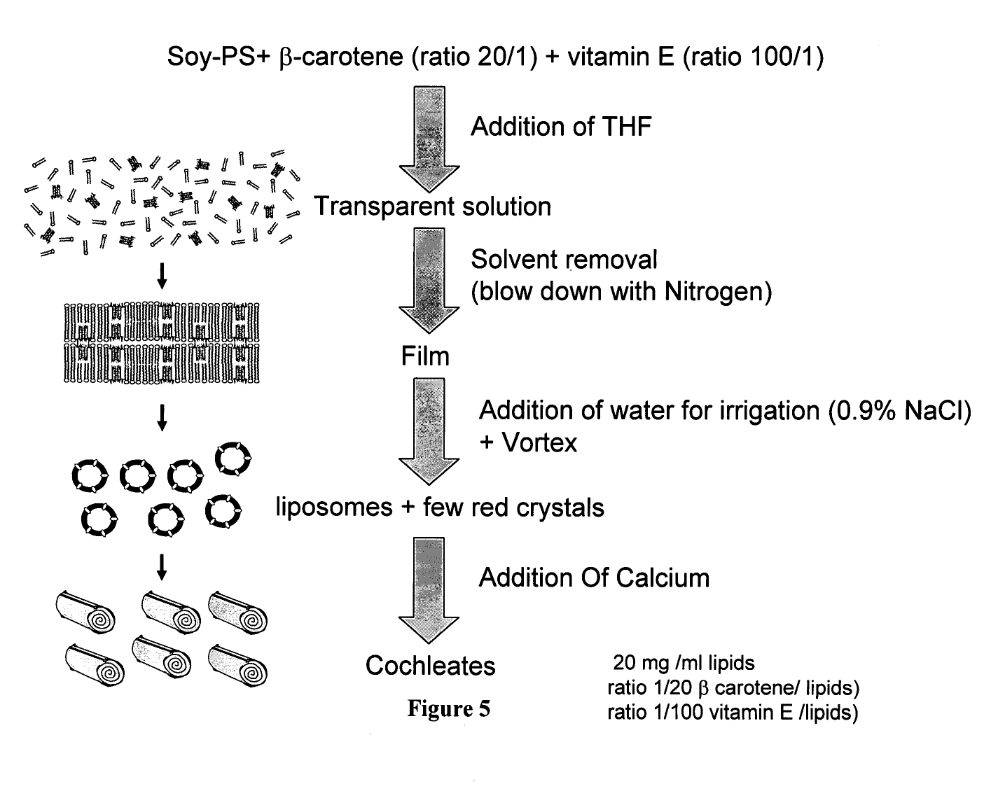

The above method is illustrated generally in FIG. 1, and more specifically in FIG. 5 for a beta-carotene and vitamin E cochleate. Referring to FIG. 1, lipid (e.g., soy PS) and fragile nutrient (e.g., beta-carotene) are mixed in a solvent (e.g., THF) at suitable ratios. The fragile nutrients can be added using this method at any ratio that allows for formation of liposomes in the following steps. The range of ratios is quite large (e.g., 1:1 to 1000:1) and thus the amount of nutrients added primarily will be determined by the desired concentration of the nutrient(s) in the cochleates. Similarly, the amount of solvent can be any amount that forms a solution that can be used for form liposomes by direct addition of a salt-water solution.

The solvent is then removed (e.g., with a nitrogen blow down) to form a lipid-fragile nutrient film. Salt-water (e.g., saline with 0.8% NaCl), is then added and the mixture vortexed to form liposomes that incorporate some or all of the fragile nutrient component. Multivalent cation is then added to form cochleates.

Upon visual inspection, it is believed that the cochleates of the invention (e.g., beta carotene or fish oil cochleates) are smaller than conventional cochleates. In one embodiment, the cochleates have a mean particle size of less then about 10 μm, preferably less than about 5 μm, 3 μm, 2 μm, 1 μm, or even 0.5 μm

Cochleates then can optionally be lyophilized using conventional methods and stored at room temperature indefinitely or can be stored in a cation-containing buffer at 40° C. for at least six months.

The method is not limited by the method of forming cochleates. Any known method can be used to form cochleates from the liposomes of the invention (i.e., the liposomes associated with the fragile nutrients). In a preferred embodiment, the cochleate is formed by precipitation. The cochleates also could be formed, e.g., by dialysis against buffered cation or any other known method. The liposome can be precipitated with a multivalent cation to form a cargo moiety-cochleate.

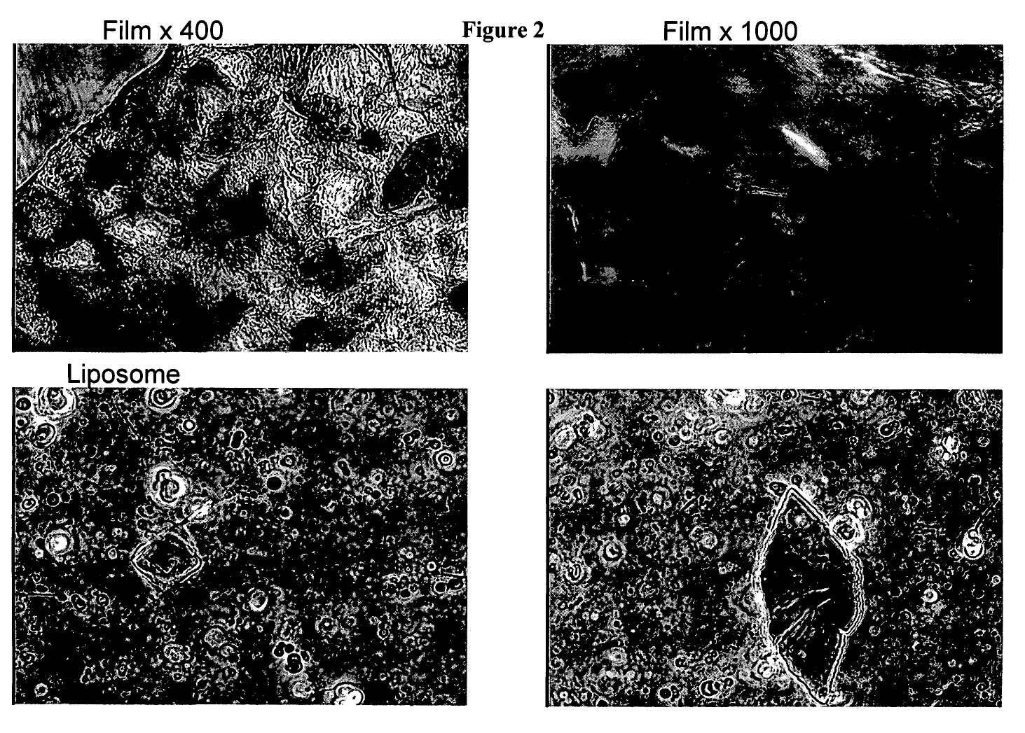

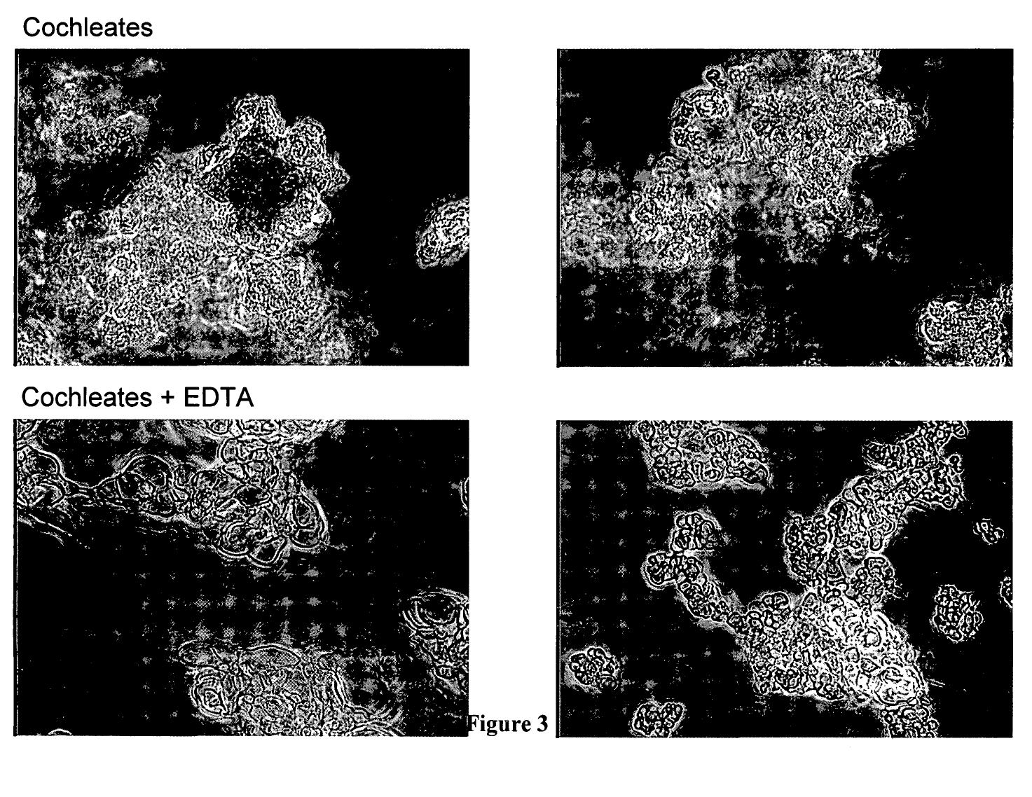

FIGS. 2 and 3 are images of beta-carotene cochleates in various stages of manufacture made according to the method described in FIG. 1. In FIG. 2, the top two images are images of a solution of soy PS, THF and beta-carotene. The darker crystals are beta-carotene which are orange-red in color and thus show up as a darker image. The bottom two images are images of a liposomal solution, that includes some free beta-carotene (dark crystals) and some beta-carotene liposomes (globular).

The top two images of FIG. 3 are images of beta-carotene cochleates. In the left image, two beta-carotene crystals (dark crystals) can also be seen encrusted into the cochleate surface (lighter globular structure). In the right image, cochleates that are darker are beta-cochleates, while lighter cochleates do not contain beta-carotene. The bottom two images of FIG. 3 are images of beta-carotene liposomes or liposome-like structures obtained by adding EDTA to open up the beta-carotene cochleates.

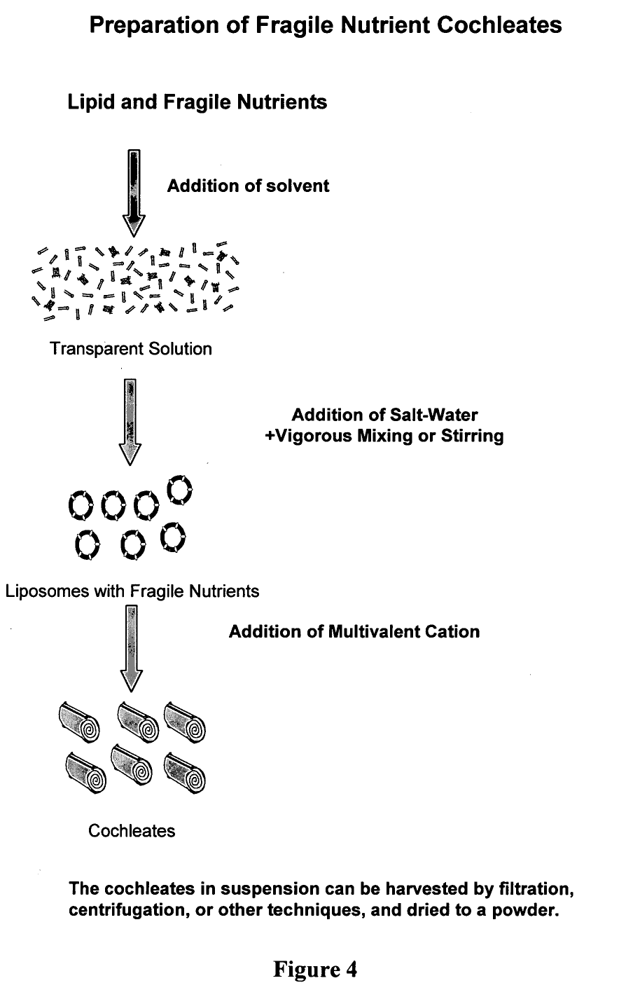

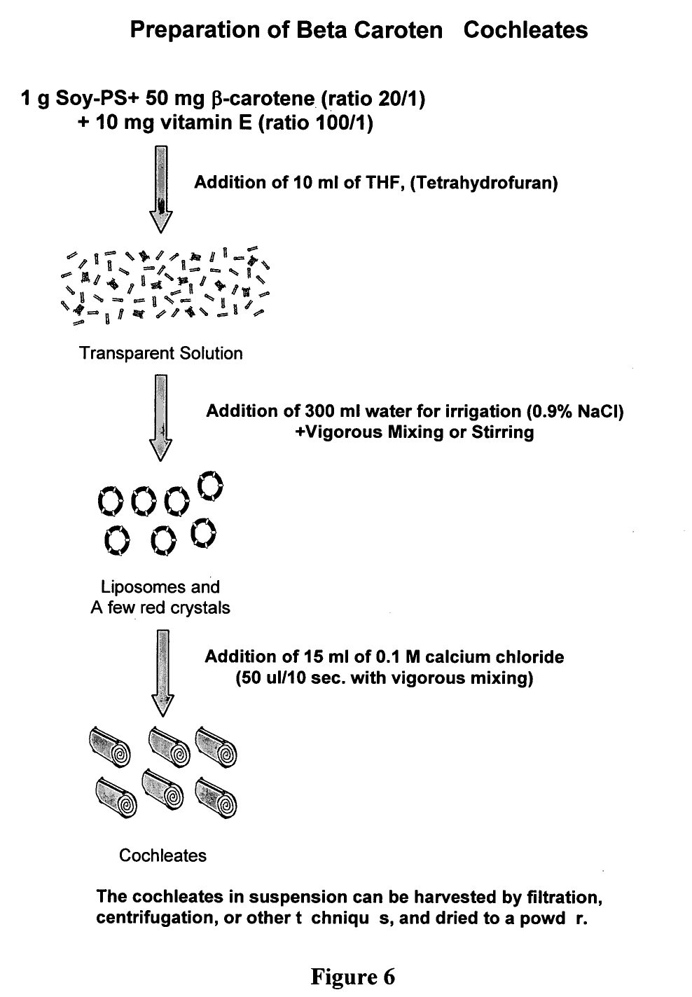

Fragile nutrient cochleates can be prepared by use of a method that includes the step of forming liposomes by direct addition of saline to a lipid/nutrient/solvent mixture to form liposomes. This method eliminates the solvent film step described in connection with FIG. 1. It is believed that the direct addition of salt water (e.g., saline) to a lipid/nutrient/solvent solution induces the formation of liposomes by sufficiently diluting out the solvent. The method used is illustrated generally in FIG. 4, and more specifically in FIG. 6 in connection with the formation of soy PS cochleates having both beta-carotene and vitamin E.

Referring to FIG. 4, a lipid (e.g., soy PS) and one or more fragile nutrients (e.g., beta-carotene and vitamin E) are mixed in a solvent (e.g., THF) at desired ratios to form a solution of lipid and nutrients in solvent. The fragile nutrients can be added using this method at any ratio that allows for formation of liposomes in the following steps. The range of ratios is quite large (e.g., 1:1 to 1000:1) and thus the amount of nutrients added primarily will be determined by the desired concentration of the nutrient(s) in the cochleates. Similarly, the amount of solvent can be any amount that forms a solution that can be used for form liposomes by direct addition of a salt-water solution.

Salt water (e.g., saline at 0.9% NaCl) is added to the lipid/nutrients/solvent solution. The amount needed to form liposomes can readily be determined by titrating the salt water solution into the lipid/nutrient/solvent solution until liposomes form. Any of various salts (e.g., calcium chloride and/or sodium chloride) in water can be used to practice this method.

Cation can them be added (e.g., 0.1 M CaCl at a rate of 50 ul/10 sec.) to form cochleates. Any multivalent cation can be utilized to form cochleates, including but not limited to, calcium, magnesium, zinc, barium, zinc, iron and other elements capable of forming ions or other structures having multiple positive charges capable of chelating and bridging negatively charged lipids. The cochleates then can optionally be harvested from the suspension by filtration, drying, centrifugation, or any of various known techniques. The cochleates can also be dried to a powder, if desired.

Formation of the cochleates of the invention in the above methods involves crystallization of multivalent cation with negatively charged lipids. It is evident, therefore, that all of the parameters that govern crystallization, e.g., temperature, lipid concentration, calcium concentrations, rate of calcium addition, and rate of mixing, can be utilized to regulate cochleate formation. Such variations can readily be manipulated by the skilled practitioner using no more than the instant specification and routine experimentation. In addition, because a cochleate is highly thermodynamically stable, once a cochleate formulation method is developed for a given product, the end product can be made predictably and reliably.

Cochleates made with different and/or combinations of cations have different structures and convert to liposomes at different rates. Because of those structural differences, the rate of release of the fragile nutrients and any other cargo moieties contained therewith varies. Accordingly, by combining cochleates made with different cations, formulations that will release the cargo moiety over a protracted period of time are obtainable.

The amount of fragile nutrient incorporated into the cochleates can be varied as desired. Because of the advantageous properties of fragile nutrient cochleates (e.g., the remarkable stability of the encochleated fragile nutrients), lesser amounts of nutrient can be used to achieve the same end result as compared to using known delivery means. The optimal lipid:fragile nutrient ratio for a desired purpose can readily be determined without undue experimentation. Various ratios are configured and the progress of precipitation of each sample is monitored visually under a phase contrast microscope. The precipitates can then be administered to the targeted host to ascertain the nature and tenor of the biologic response to the administered cochleates. It is evident that the optimized ratio for any one use may range from a high ratio to a low ratio to obtain maximal amount of cargo moiety in the cochleates. The cochleate formulations also can be prepared both with and without targeting molecules (e.g., fusogenic molecules, such as Sendai virus envelope polypeptides), to target specific cells and/or tissues.

The cochleates of the present invention are surprisingly heat and pressure stable, such that a wide variety of processing methods and conditions can be used to process the cochleates and anything to which they are added. By way of example, beta-carotene cochleates can be autoclaved even above 160° C. and 400 psi without degradation of beta-carotene. The cochleates of the present invention can be subjected to a wide variety of food processing methods, e.g., agglomeration, steaming, drying (e.g., air drying, oven drying, spray drying and drum drying), microwaving, calendaring, mixing, filtration, vortexing, and baking, without degradation of the encochleated fragile nutrient. It has been observed that non-encochleated beta-carotene will degrade over time or when subjected to such processing (which can be indicated by a slight color change), but encochleated beta-carotene will not.

Another striking example of the ability of the fragile nutrient cochleates of the present invention to prevent the degradation of the encochleated species is described, for example, in Example 3. In this example, beta-carotene cochleates are incorporated into a muffin batter and baked at 425° C. The beta-carotene in the resulting muffins was not degraded by the elevated temperature as indicated by the intense red-orange color observed.

In yet another aspect, the invention features a method of delivering the one or more fragile nutrients and optional additional cargo moieties to a subject. The method includes administering to a subject a biologically effective and/or nutritionally supplemental amount of the cochleates of the invention. The cochleates and cochleate compositions of the present invention can be administered to animals, including both human and non-human animals. It can be administered to animals, e.g., topically or in animal feed or water.

Accordingly, in yet another aspect, the invention provides a method of delivering fragile nutrients to a subject comprising administering to a subject a biologically effective amount of fragile nutrient cochleate. The fragile nutrient cochleate can be delivered in the form of a food item. The food item can be a health bar, snack food, dog food, cat food, animal food, or health drink.

Another advantage of the cochleates of the present invention is the stability and safety of the composition, particularly when soy-based lipids are employed. Thus, the cochleates can be administered orally or by instillation without concern, as well as by the more traditional routes, such as mucosal, systemic, topical, subcutaneous, intradermal, transdermal, intranasal, intraocular, intrarectal, intravaginal, intrapulmonary, intravenous, intramuscular, and the like. Direct application to mucosal surfaces is an attractive delivery means made possible with the cochleates of the invention.

In certain embodiments, the fragile nutrient cochleate includes an additional cargo moiety. Accordingly, the present invention provides a method of treating a subject that would benefit from the administration of a cargo moiety and/or a fragile nutrient and/or a fragile cargo moiety. The benefit can be treatment of a disease or disorder.

The disease or disorder can be, e.g., inflammation, pain, infection, fungal infection, bacterial infection, viral infection, parasitic disorders, an immune disorder, genetic disorders, degenerative disorders, cancer, proliferative disorders, obesity, depression, hair loss, impotence, hypertension, hypotension, dementia, senile dementia, or malnutrition.

“Treatment”, or “treating” as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. “Treated,” as used herein, refers to the disease or disorder being cured, healed, alleviated, relieved, altered, remedied, ameliorated improved or affected.

The above methods can be employed in the absence of other treatment, or in combination with other treatments. Such treatments can be started prior to, concurrent with, or after the administration of the compositions of the instant invention. Accordingly, the methods of the invention can further include the step of administering a second treatment, such as for example, a second treatment for the disease or disorder or to ameliorate side effects of other treatments. Such second treatment can include, e.g., radiation, chemotherapy, transfuision, operations (e.g., excision to remove tumors), and gene therapy. Additionally or alternatively, further treatment can include administration of drugs to further treat the disease or to treat a side effect of the disease or other treatments (e.g., anti-nausea drugs).

The skilled artisan can determine the most efficacious and therapeutic means for effecting treatment practicing the instant invention. Reference can also be made to any of numerous authorities and references including, for example, “Goodman & Gilman's, The Pharmaceutical Basis for Therapeutics” (6th Ed., Goodman et al., MacMillan Publ. Co., New York, 1980).

In addition, the U.S. Recommended Daily Intake (USRDI) for vitamins and minerals are defined and set forth in the Recommended Daily Dietary Allowance-Food and Nutrition Board, National Academy of Sciences-National Research Council. As used herein, a nutritionally supplemental amount of minerals other than iron or zinc is at least about 5%, preferably from about 10% to about 200%, of the USRDI of such minerals. As used herein; a nutritionally supplemental amount of vitamins is at least about 5%, preferably from about 20% to about 200%, more preferably from about 25% to 100%, of the USRDI of such vitamins.

Current USRDI values for most healthy adults are generally: vitamin C (60 mg), vitamin A (1 mg), beta-carotene (3 mg), vitamin B₂ (1.7 mg), niacin (20 mg), thiamin (1.5 mg), vitamin B6 (2.0 mg), folic acid (0.4 mg), vitamin B₁₂ (6 μg), and vitamin E (30 international units) and for iodine is 150 μg.

It is recognized, however, that the preferred daily intake of any vitamin or mineral may vary with the user. For example, persons suffering with anemia may require an increased intake of iron. Persons suffering vitamin deficiencies or who have poor diets will require more nurtrients, particularly growing children in developing countries. Such matters are familiar to physicians and nutritional experts, and usage of the compositions of the present invention may be adjusted accordingly.

Nutritional and/or pharmaceutical formulations can be of solid form including tablets, capsules, pills, bulk or unit dose powders and granules or of liquid form including solutions, fluid emulsions, fluid suspensions, semisolids and the like. In addition to the active ingredient, the formulation would comprise relevant molecules, suitable art-recognized diluents, carriers, fillers, binders, emulsifiers, surfactants, water-soluble vehicles, buffers, solubilizers and preservatives. Formulations incorporating the cochleates of the present invention can be of liquid or semi-liquid form including food products, such as therapy or nutrient drinks, yogurt, milk, salad dressing, moist animal food, and the like.

The cochleates of the instant invention can be particularly advantageous for delivering fragile nutrients and other agents to food and drinks to be consumed by humans or other animals. For example, animal food (e.g., human, cat, dog, fish, and bird food), can include the cochleates of the present invention to stably deliver vitamins, minerals or other nutrients, as well as medications, e.g., allergy medications and/or additional cargo moieties. The fragile nutrient cochleate formulations of the present invention can be added to pet or domestic animal feed, such as fish food and food for fowl, cattle, and horses. The vehicles can be added at any step of the preparation. For example, the formulations of the invention can be added at any point in the methods described in WO 02/44026, incorporated herein by this reference. Similarly, the compositions and methods of the invention can be employed in food or drink to be consumed by humans, e.g., in nutrient bars or drinks, cereals, breads, and snack foods. Accordingly, the preparations of the invention allow for the production of stable, convenient preparations of micronutrients in processed foods, such as fast foods. Typically, potentially beneficial micronutrients, e.g., omega fatty acids and antioxidants, can be destroyed during food manufacture and storage. The formulations of the invention protect micronutrients and other cargo moieties, thus increasing the nutritional and/or medicinal value of the food.

Because of their increased stability, the compositions and methods of the present invention are particularly useful in foods that are baked or cooked, such as cakes, muffins, pasta noodles, soups, cereals, chips, candy and cookies. In a preferred embodiment, the compositions are used in candy, such as candy bars, e.g., chocolate bars. For example, omega fatty acid-cochleates can be incorporated into a chocolate bar.

The fragile nutrient cochleate formulations of the invention can be added to food items, e.g., fast food products, in the crystallized or emulsion form at any stage of the manufacturing process. The food item can be an animal food item, a human food item, a nutrient bar, a snack food, a beverage, a domesticated animal food, a fish food, a poultry feed, a pet food, a dog food or a cat food.

Use of the formulations of the present invention, e.g., fragile nutrient cochleates, can result in an increase in the amount of active ingredient delivered versus that which can be achieved with conventional food or drug preparations. For example, the delivery vehicles of the present invention can result in a 20%, 40%, 50%, 60%, 100%, 200% . . . 1000% . . . 10,000% increase in the active (undegraded) ingredient delivered versus use of the cargo directly in the preparation of the drug, food, beverage, etc.

The present invention is useful in a variety of foods, including, dried food and beverage mixes, ready-to-drink and eat beverages and foods. These include baked good mixes and baked goods (e.g., bread, cakes, brownies, muffins, cookies, pastries, pies, crackers, pie crusts), fried snacks derived from potatoes, corn, wheat and other grains (e.g., potato chips, corn chips, tortilla chips), other fried farinaceous snack foods (e.g., french fries, doughnuts, fried chicken), dairy products and artificial dairy products (e.g., butter, ice cream and other fat-containing frozen desserts, yogurt, and cheeses, including natural cheeses, processed cheeses, cream cheese, cottage cheese, cheese foods and cheese spread, milk, cream, sour cream, butter milk, and coffee creamer), cereal products, baby foods or formulas, puddings, ice cream, dips, syrups, pie and other dessert fillings, frostings, emulsified spreads such as salad dressings, mayonnaise and margarines, various kinds of soups, dips, sauces and gravies. The preparations can include additional agents typically found in food preparations, such as coloring agents, flavoring agents, edible acids, preservatives, and the like.

The cochleates can be added to the food products in a crystallized or emulsion form at any stage of the manufacturing process. The cochleates can be added at a stage and in a manner where the integrity of the delivery vehicle is maintained until ingestion, or final preparation of the food product by the consumer. Another alternative, however, can be to use the cochleates to maintain the stability of the agent until incorporation into the product, so activity can be maintained during storage and shipping. For example, food and drink mixes can contain cochleates that de-precipitate when reconstituted prior to ingestion. In this case, the cochleates maintain the stability and integrity of the fragile nutrient until ingestion so that the ingested food or drink contains the fragile nutrient in a non-degraded state.

Yet another alternative is to deliver the formulations themselves to consumers or professionals, for direct addition to food products, e.g., medicament, nutrient crystals, additives, supplements, or emulsions, such that the user can vary the concentration as desired.

The fragile nutrient cochleate formulations can also be added to a carrier for use as a topical treatment on the skin. Suitable carriers would remain on the skin for an extended period of time, and be resistant to perspiration or immersion in water. Thus, for example, the formulations may be added to topical applications of medicaments, moisturizers, deodorants, balms, fragrances, sunscreens, and the like.

Additional examples of formulations that can include the cochleates of the invention include, but are not limited to, hair care products, skin care products, personal care products, personal cleansing products, lotions, fragrances, sprays, perfumes, cosmetics, toothpastes, tooth whiteners, cleaners, bar soap, liquid soap, body wash, baby wash, makeup, hair color, shampoos, conditioners, styling products, balms, creams, solutions, gels and solids. Thus, for example, shampoos, conditioners and the like may contain cochleates loaded with vitamins, moisturizers, perfumes, medications, etc.

The cochleates of the invention can also be added to cleansers which do not have direct contact with the skin. These formulations would be advantageous for, i.e., the incorporation of perfumes, moisturizers or other such cargo moieties into fabric or for the introduction of an antibacterial agent to dishes. Examples include, but are not limited to, laundry detergent, pre-treating formulations, dryer sheets, fabric softener, and dishwashing detergent.

Cochleates of the present invention can also be added to paper products for the topical application of cargo moieties to skin. Examples of paper products that can include cochleates of the invention include baby care products, i. e, diapers or baby wipes, tissues, toilet paper, antibacterial or antiperspirant towelettes, napkins, paper towels, bandaids, gauze pads, and feminine hygiene products.

In yet another aspect, the invention provides an article of manufacture of cochleate formulations of the invention. The article of manufacture includes packaging material and a lipid contained within the packaging material. The packaging material includes a label or package insert indicating the use of the lipid for forming cochleate formulations of the invention. The article of manufacture can further include instructions or guidelines for the formation of cochleate formulations of the invention, e.g., mixing the lipid and a fragile nutrient with a solvent and adding it to an aqueous solution. Optionally, the article of manufacture can include a solvent, a nutrient, a multivalent cation (e.g., calcium and/or magnesium), a cargo moiety, and/or a chelating agent (e.g., EDTA). The article of manufacture may further include other ingredients or apparatus that can be employed to manufacture the compositions of the present invention.

In another aspect, the invention provides a cochleate formulation of the present invention packaged with instructions for adding the vehicle to a food, beverage or personal care product.

In yet another aspect, the present invention provides a fragile cargo moiety cochleate formulation for delivery of a fragile cargo moiety that is susceptible to degradation by, e.g., air, water and light. The cochleate generally includes a fragile cargo moiety component, and a cochleate comprising a negatively charged lipid component and a multivalent cation component.

In another aspect, the invention provides a method of making a fragile cargo moiety cochleate formulation. The method includes the steps of (i) dissolving a negatively-charged lipid component and a fragile cargo moiety component in an organic solvent to form a solution; (ii) forming fragile cargo moiety liposomes from the solution; and (iii) exposing the fragile cargo moiety liposomes to a multivalent cation to form fragile cargo moiety cochleates.

In yet another aspect, the present invention provides a method of delivering a fragile cargo moiety to a subject, the method comprising the step of: administering to a subject a biologically effective amount of fragile cargo moiety cochleate. The cochleates can be administered in any of the forms and using any of the methods described herein.

In certain embodiments, the fragile cargo moiety in the cochleates and methods described herein is selected from the group consisting of an amino acid, a toxin, a microbicide, a microbistat, a co-factor, an enzyme, a polypeptide, a polypeptide aggregate, a polynucleotide, a lipid, a carbohydrate, a nucleotide, a starch, a pigment, a fatty acid, a monounsaturated fatty acid, a polyunsaturated fatty acid, a flavor substance, a flavored essential oil or extract, a hormone, a cytokine, a virus, an organelle, a steroid or other multi-ring structure, a saccharide, a metal, a metabolic poison, an antigen, an imaging agent, a porphyrin, a tetrapyrrolic pigment, or a drug. The fragile cargo moiety preferably comprises at least 1%, more preferably at least 2%, and more preferably at least 5% of the formulation by weight. Preferably, the fragile cargo moiety is substantially incorporated into the cochleate. Any of the methods, solvents, cations, lipids, etc. described herein can be used to form the fragile cargo moiety cochleates. The fragile cargo moiety cochleates may also further include additional cargo moieties, fragile cargo moieties and/or fragile nutrients, as described herein.

The fragile cargo moiety cochleates can be administered to a subject to treat inflammation, pain, infection, fungal infection, bacterial infection, viral infection, parasitic disorders, an immune disorder, genetic disorders, degenerative disorders, cancer, diabetes, insomnia, proliferative disorders, obesity, depression, hair loss, impotence, hypertension, hypotension, dementia, senile dementia, or malnutrition.

Practice of the invention will be still more fully understood from the following examples, which are presented herein for illustration purposes only and should not be construed as limiting the invention in any way. cl EXEMPLIFICATION

### Example 1

### Formation of a Fragile Nutrient Cochleate

Cochleate preparations of beta-carotene and vitamin E were made as illustrated generally in FIG. 1 and specifically in FIG. 5. Soy phosphatidylserine (soy PS), beta-carotene (in a 20:1 ratio), and vitamin E in (in a 100:1 ratio of lipid to vitamin E) were mixed. The solvent, tetrahydrofuran (THF), was added to this mixture to achieve a solution of lipid, beta-carotene, vitamin E, and THF. The solvent was subsequently removed with a nitrogen blow down to form a lipid/beta-carotene/vitamin E film. Salt-water (0.9% NaCl) was subsequently added and the mixture vortexed to form liposomes that had beta-carotene, which can be visually identified as red crystals, in the liposomal bilayers. Calcium was then added to form an encochleated beta-carotene and vitamin E preparation.

### Example 2

### Formation of a Fragile Nutrient Cochleate

Approximately 1 g of soy PS, 50 mg beta-carotene (ratio 20:1 ratio), and 10 mg vitamin E (100:1 ratio) were mixed. About 10 ml of THF solvent was added to this mixture and a solution of lipid, beta-carotene, vitamin E, and THF was achieved. About 300 ml of saline (water with 0.9% NaCl) was added for irrigation and the mixture was vigorously mixed or stirred to form liposomes having had beta-carotene (red crystals) in the liposomal bilayers. Approximately 15 ml of 0.1 M calcium chloride was added at a rate of 50 ul/10 sec. with vigorous mixing to form cochleates in suspension. The method used is illustrated generally in FIG. 4 and specifically for beta-carotene and vitamin E in FIG. 6. The cochleates were then harvested by drying the suspension in an oven at 450° C.

### Example 3

### Food Items Prepared With and Without Fragile Nutrient Cochleates

Beta-carotene cochleates made in accordance with the present invention were added to a blueberry muffin mix and baked at approximately 425° F. for about 15 minutes. Muffins also were made without beta carotene-cochleates under the same conditions as a control. The reddish beta-carotene color of the cochleates persisted even after cooking, indicating that the encochleated beta-carotene was well preserved.

### Example 4

### Formation of Fish Oil Cochleates

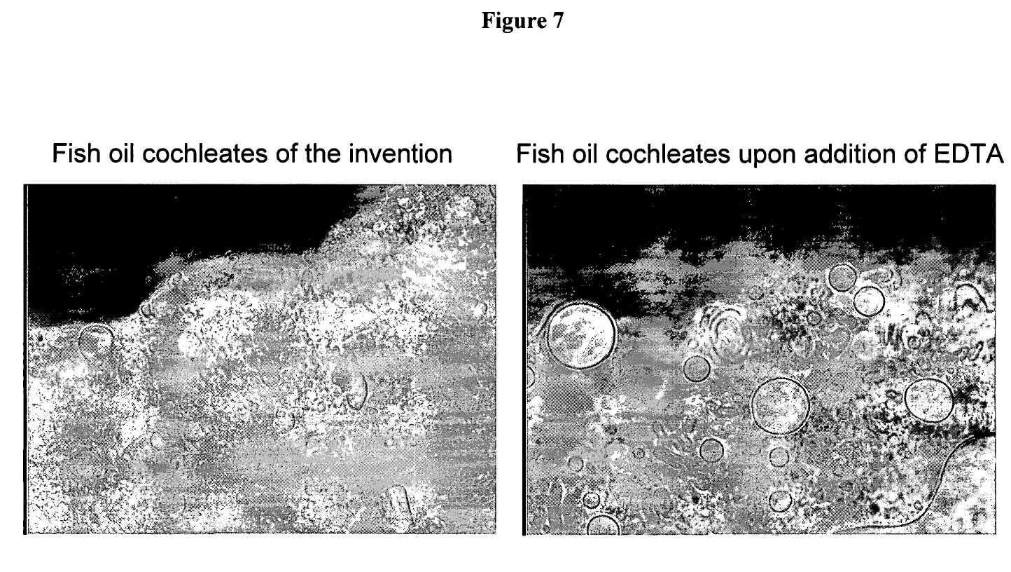

0.8 g of Soy-PS (Chemi) and 0.2 g of Fish oil (Marine) were completely dissolved in 5 ml of THF. 50 ml of water was then added to the solution and stirred vigorously. 2 ml of 2.5M of Calcium Chloride was added to the aqueous mixture in order to form cochleates. FIG. 7 shows two images of fish oil cochleates as viewed under a microscope before (left image) and after (right image) addition of a chelating agent (EDTA). Upon addition of EDTA, the cochleates open and release their contents. Liposomes and large fish oil droplets resulting from the opening of the cochleates can be visualized in FIG. 7. These images indicate a substantial amount of fish oil was incorporated into the cochleates of the present invention.

### Example 5

### Preparation of Beta-carotene Cochleates with Vitamin E

5 g of beta-carotene was added to 390 ml of THF, and the solution was stirred at medium speed until the beta-carotene was completely dissolved. 100 g of Soy-PS (Degussa) was added in small quantities, and the solution was continuously stirred until the Soy-PS was completely dissolved. 1 g of vitamin E (Roche) was dissolved it in 10 ml of THF, and this solution was subsequently added to the Soy-PS/beta-carotene solution. With stirring, 2000 ml of saline was added to the solution. The solution was observed to become cloudy and orange with few crystals of beta-carotene on the surface. While continuing to stir, 8 g calcium chloride was added slowly to the mixture. Upon addition of all of the calcium chloride, cochleates were observed to float on the surface. The solution was slowly filtered under vacuum and washed with 1000 mL of washing buffer to remove the residual THF. The product was then dried for 24 to 48 hours in an oven at 45C.

Equivalents

Those skilled in the art will recognize, or be able to ascertain using no more than routine experimentation, many equivalents to the specific embodiments of the invention described wherein. Such equivalents are intended to be encompassed by the following claims. For example, applications and formulations including the fragile nutrient liposomes described herein are intended to be within the scope of the present invention.

1. A fragile nutrient cochleate formulation comprising:
a fragile nutrient component; and
a cochleate comprising a negatively charged lipid component and a multivalent cation component.

2. The formulation of claim 1, comprising at least 1% fragile nutrient component by weight.

3. The formulation of claim 1, comprising at least 3% fragile nutrient component by weight.

4. The formulation of claim 1, comprising at least 5% fragile nutrient component by weight.

5. The formulation of claim 1, wherein the fragile nutrient component is substantially incorporated into the cochleate structure.

6. The formulation of claim 1, wherein the negatively charged lipid component comprises phosphatidylserine.

7. The formulation of claim 1, wherein the negatively charged lipid component comprises a soy phosphatidylserine.

8. The formulation of claim 1, wherein the fragile nutrient is a phytochemical.

9. The formulation of claim 1, wherein the fragile nutrient is an antioxidant phytochemical.

10. The formulation of claim 1, wherein the fragile nutrient is at least one phytochemical selected from the group consisting of: beta-carotene, lutein, zeaxanthine, quercetin, silibin, perillyl alcohol, genistein, sulfurophane and lycopene.

11. The formulation of claim 1, wherein the fragile nutrient is a zoochemical.

12. The formulation of claim 1, wherein the fragile nutrient is at least one zoochemical selected from the group consisting of: omega-3 and omega-6 fatty acids.

13. The formulation of claim 1, further comprising one or more additional cargo moiety.

14. The formulation of claim 1, comprising at least one additional cargo moiety selected from the group consisting of: a vitamin, a mineral, a nutrient, a micronutrient, an amino acid, a toxin, a microbicide, a microbistat, a co-factor, an enzyme, a polypeptide, a polypeptide aggregate, a polynucleotide, a lipid, a carbohydrate, a nucleotide, a starch, a pigment, a fatty acid, a monounsaturated fatty acid, a polyunsaturated fatty acid, a flavor substance, a flavored essential oil or extract, a hormone, a cytokine, a virus, an organelle, a steroid or other multi-ring structure, a saccharide, a metal, a metabolic poison, an antigen, an imaging agent, a porphyrin, a tetrapyrrolic pigment, and a drug.

15. The formulation of claim 1, wherein the fragile nutrient component is beta-carotene and the formulation further comprises a vitamin E component.

16. A pharmaceutical composition comprising a fragile nutrient cochleate formulation of any of claims 1-15 and a pharmaceutically acceptable carrier.

17. A food item comprising the fragile nutrient cochleate formulation of any of claims 1-15.

18. The food item of claim 17, wherein the food item is selected from the group consisting of: health bars, snack foods, domesticated animal foods, fish foods, poultry feeds, dog foods, cat foods, animal foods, and health drinks.

19. A personal care product comprising the fragile nutrient cochleate formulation of any of claims 1-15.

20. The personal care product of claim 19, wherein the personal care product is a hair care product or a skin care product.

21. A method of making a fragile nutrient cochleate formulation, the method comprising the steps of:
dissolving a negatively-charged lipid component and a fragile nutrient component in an organic solvent to form a solution;
forming fragile nutrient liposomes from the solution; and
exposing the fragile nutrient liposomes to a multivalent cation to form fragile nutrient cochleates.

22. The method of claim 21, wherein the step of forming the fragile nutrient liposomes includes the addition of salt water to the solution.

23. The method of claim 21, wherein the step of forming the fragile nutrient liposomes includes removal of the solvent from the solution to form a film.

24. The method of claim 21, wherein the solvent comprises a solvent selected from the group consisting of tetrahydrofuran, chloroform, dichloromethane, carbon tetrachloride, butanol, hexane, ethanol, toluene, benzene, ether, petrol ether, oil and combinations thereof.

25. The method of claim 21, further comprising the step of adding the fragile nutrient cochleate to a food item.

26. The method of claim 25, wherein the food item is selected from the group consisting of: health bars, snack foods, domesticated animal foods, fish foods, poultry feeds, dog foods, cat foods, animal foods, and health drinks.

27. The method of claim 21, further comprising the step of adding the fragile nutrient cochleate to a personal care product.

28. The method of claim 27, wherein the personal care product is a hair care product or a skin care product.

29. A method of delivering fragile nutrients to a subject, the method comprising the step of: administering to a subject a biologically effective amount of fragile nutrient cochleate.

30. The method of claim 29, wherein the fragile nutrient cochleate is delivered in the form of a food item.

31. The method of claim 30, wherein the food item is selected from the group consisting of: a health bar, a snack food, a beverage, a domesticated animal food, a fish food, a poultry feed, a dog food, a cat food, an animal food, and a health drink.

32. The method of claim 29, wherein the fragile nutrient cochleate is delivered in the form of a personal care product.

33. The method of claim 32, wherein the personal care product is a hair care product or a skin care product.

34. The method of claim 29, wherein the administration route is selected from the group consisting of mucosal, systemic, oral, intranasal, intraocular, intrarectal, intravaginal, intrapulmonary, intravenous, intramuscular, subcutaneous, transdermal and intradermal.

35. The method of claim 29, wherein the cochleate comprises an additional fragile nutrient.

36. The method of claim 29, wherein the cochleate comprises at least one additional cargo moiety.

37. The method of claim 36, wherein the at least one additional cargo moiety selected from the group consisting of: a vitamin, a mineral, a nutrient, a micronutrient, an amino acid, a toxin, a microbicide, a microbistat, a co-factor, an enzyme, a polypeptide, a polypeptide aggregate, a polynucleotide, a lipid, a carbohydrate, a nucleotide, a starch, a pigment, a fatty acid, a monounsaturated fatty acid, a polyunsaturated fatty acid, a flavor substance, a flavored essential oil or extract, a hormone, a cytokine, a virus, an organelle, a steroid or other multi-ring structure, a saccharide, a metal, a metabolic poison, an antigen, an imaging agent, a porphyrin, a tetrapyrrolic pigment, and a drug.

38. The method of claim 37, wherein the fragile nutrient cochleate formulation is administered to treat inflammation, pain, infection, fungal infection, bacterial infection, viral infection, parasitic disorders, an immune disorder, genetic disorders, degenerative disorders, cancer, diabetes, insomnia, proliferative disorders, obesity, depression, hair loss, impotence, hypertension, hypotension, dementia, senile dementia, or malnutrition.
